(19) United States
(12) Patent Application Publication
(10) Pub. No.: US 2005/0059994 A1
(43) Pub. Date: Mar. 17, 2005

(54) Fatigue resistant medical devices

(76) Inventors: Steven Walak, Natick, MA (US); Yixin Xu, Newton, MA (US)

Correspondence Address: Patrick J. Fay, Esq.;FAY KAPLUN & MARCIN, LLP, Suite 702, 150 Broadway, New York, NY, 10038, US

(21) Appl. No.: 10/666,863

(22) Filed: Sep. 17, 2003

(51) Int. Cl. A61F 2/06

(52) U.S. Cl. 606/200; 606/78; 623/1.19

(57) ABSTRACT

A flexible device comprises a metallic element including high strain portions and lesser strain portions, wherein the high strain portions are to be subjected to levels of strain during use increased with respect to strain levels in the lesser strain portions. The high strain portions comprise a material which, under predetermined operating conditions, is stabilized in a martensite phase and the lesser strain portions comprise a material which, under the predetermined operating conditions, is in an austenite phase. A method of forming an element of a medical device comprises the steps of forming an element of the device of Nitinol and impressing a memorized shape on the element, wherein the memorized shape is a shape the element is to assume when in an operational configuration. A high strain portion of the element is treated so that it is substantially Martensite phase stabilized under expected operating conditions of the device, wherein untreated portions of the element are in a substantially austenitic phase under the expected operating conditions.

### FIELD OF THE INVENTION

The present invention is related generally to devices adapted to be implanted in a patient's body and which are subject to cyclic strains. In particular, the present invention is related to devices used in a patient's vascular system, which retain a specified shape when deployed.

### BACKGROUND OF THE INVENTION

Modern medical procedures used to treat vascular diseases routinely employ implants placed in a patient's vascular system to perform various therapeutic functions. As an example, some courses of treatment to open blocked arteries may include inserting a stent in the artery to maintain a flow lumen of the artery open at a location from which a blockage has previously been removed, for example during a balloon angioplasty procedure. In another example, treatment for pulmonary embolism may include the removal of blood clots from a patient's blood stream by introducing a clot filter into the patient's vena cava which, when deployed, prevents clots from traveling through the vascular system.

Many implants used in medical procedures are inserted into the body in a folded or compact insertion configuration, to minimize discomfort to the patient and to make the procedure as non-invasive as possible. Once such a medical implant is in a position within the patient's body at which it is to be placed, it may then be deployed or expanded to an operational configuration which may be considerably larger than its insertion configuration. Such implants must therefore be able to be reliably deployed from the insertion to the operational configuration once the desired position within the patient is reached. Various elastic materials and components have been used to achieve this capability, with springs, coils and flexible elements being employed by a large portion of such expandable medical implants. These elastic components often deploy the medical implant to the operational configuration and maintain it in this configuration throughout the life of the implant.

Many of the medical implants described above take advantage of the properties of Nitinol, a nickel and titanium alloy, to achieve and maintain their deployed shape within the patient's body. Nitinol (an acronym for Nickel Titanium Naval Ordinance Laboratory) exhibits several useful properties such as a shape memory effect, by which a Nitinol component returns to a previously memorized shape after being forced into a second shape. Nitinol also exhibits superelasticity, meaning that a Nitinol component may be deformed elastically to a very large extent by strain without reducing its ability to return to the its original shape after the strain has been removed. One drawback of Nitinol, however, is that in certain configurations it is not very resistant to fatigue, i.e. repeated cyclic strains.

### SUMMARY OF THE INVENTION

The present invention is directed to a flexible device comprising a metallic element including high strain portions and lesser strain portions, wherein the high strain portions are to be subjected to levels of strain during use increased with respect to strain levels in the lesser strain portions. The high strain portions comprise a material which, under predetermined operating conditions, is stabilized in a martensite phase and the lesser strain portions comprise a material which, under the predetermined operating conditions, is in an austenite phase.

The present invention is further directed to a method of forming an element of a medical device comprising the steps of forming an element of the device of Nitinol and impressing a memorized shape on the element, wherein the memorized shape is a shape the element is to assume when in an operational configuration. A high strain portion of the element is treated so that it is substantially Martensite phase stabilized under expected operating conditions of the device, wherein untreated portions of the element are in a substantially austenitic phase under the expected operating conditions.

### BRIEF DESCRIPTION OF THE DRAWINGS

FIG. 1 is a diagram showing an exemplary embodiment of the present invention forming a vena cava filter;

FIG. 2 is a diagram showing Nitinol transition temperatures between martensite and austenite phases;

FIG. 3 is a diagram illustrating the shape memory effect of Nitinol;

FIG. 4 is a diagram illustrating the superelasticity effect of Nitinol;

FIG. 5 is a cross sectional view of a Nitinol element with a surface portion according to an embodiment of the present invention;

FIG. 6 is a cross sectional view of a Nitinol element with a surface portion according to another embodiment of the present invention; and

FIG. 7 is a cross sectional view of a Nitinol element with a surface portion according to a further embodiment of the present invention.

### DETAILED DESCRIPTION

The present invention may be further understood with reference to the following description and the appended drawings, wherein like elements are referred to with the same reference numerals.

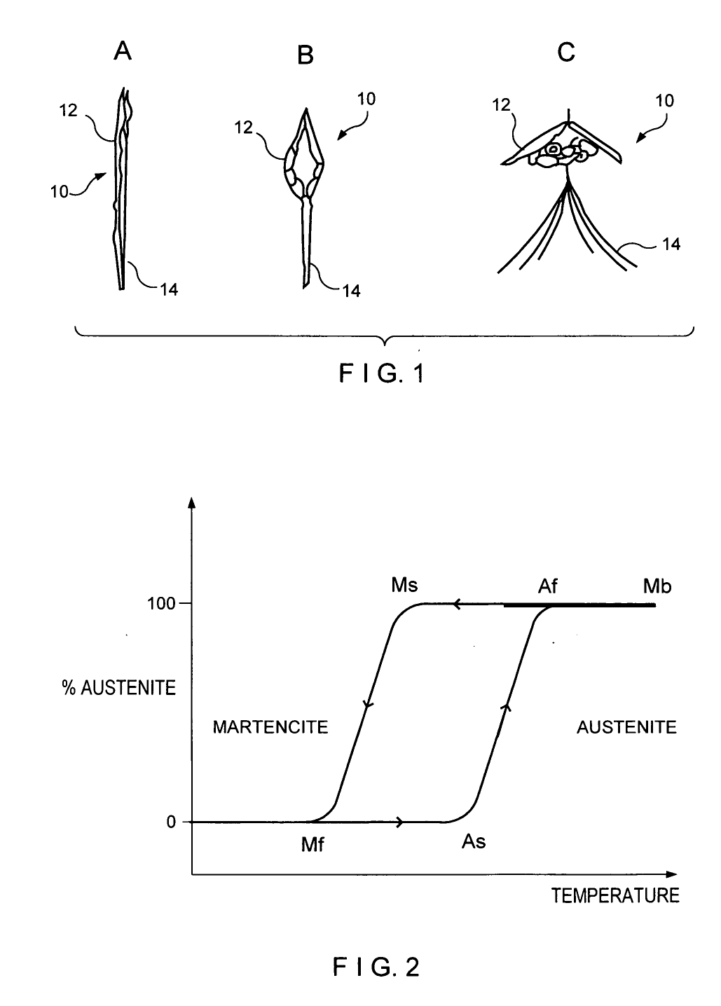

FIG. 1 shows an exemplary embodiment of a vena cava filter 10 folded into an insertion configuration A for insertion into a patient's vein. When in configuration A, a diameter of the filter 10 is substantially reduced so it may be easily inserted into a catheter extending through the patient's vascular system to a placement position (e.g., within the vena-cava).

Once a catheter containing filter 10 has reached the placement position within the vena cava, the filter 10 is deployed from the distal end of the catheter and constraint on the diameter of the filter 10 is released so that the filter 10 expands to its operational configuration, as shown in configurations B and C of FIG. 1. As the filter 10 expands from the configuration A to configuration B and, then to the fully deployed operational configuration C, a low turbulence mesh screen 12 and a plurality of hooks 14 are extended from the folded body of the filter 10. In the operational configuration, a mesh 12 of the filter 10 is spread out to trap any blood clots passing through the vena cava and hooks 14 are extended to anchor the assembly in place at the desired location within the vena cava. To extend the device from the insertion configuration A to the operational configuration C, springs or spring-like elements are included in the filter 10 which are biased to deploy the mesh 12 and the hooks 14 once the filter 10 has left the catheter. In one example, Nitinol may be used to construct all or part of the filter 10, since this material exhibits properties that are well suited for the required performance.

As described above, Nitinol is an alloy which was developed to achieve improved elasticity and other enhanced mechanical properties. Nitinol also possesses shape memory properties that are well suited for application in medical implants. Elements constructed of Nitinol may be formed in a first “memorized” shape to which they will return after a deformation. That is, when such a Nitinol element has been deformed, raising a temperature of the element above a critical temperature causes the element to revert to its memorized shape. The filter 10 may be formed including structural members of Nitinol so that the shape of the filter 10 in the configuration C is the shape “memorized” by the alloy. Thus, these elements may be formed into the shape corresponding to the configuration C while at a temperature above a critical temperature of Nitinol. The elements are then cooled below the critical temperature and deformed into shapes corresponding to the configuration A, consists, for example, of substantially straight wires that are easy to load into a catheter. Once the filter 10 has reached its operating position, it is warmed by the patient's blood and, when temperature of the filter 10 surpasses the critical temperature, these elements resume their memorized shapes to extend the filter 10 to its operating configuration C.

Nitinol has been used to manufacture other types of implantable medical devices in addition to vena cava filters. Nitinol is hypoallergenic and compatible with biological tissues, and thus can be left within the human body for extended periods of time. Vascular stents, for example, have been made of Nitinol. Similarly to the vena cava filters described above, Nitinol vascular stents may be compressed to an initial small diameter configuration for insertion into a patient's artery via a catheter. Once such a stent is in place, it may be deployed to its operational configuration. The mechanical properties of Nitinol assist in this deployment and in maintaining a desired shape of the stent as it is subject to bending and flexure in the body. In addition to stents and filters, structural components that give shape to catheters and to innumerable other devices may be manufactured of Nitinol, to take advantage of the alloy's unique properties.

As would be understood by those of skill in the art, Nitinol alloys can exist in one of two different temperature-dependent crystal structures. At lower temperatures, Nitinol is martensitic, meaning that its structure is composed of self-accommodating twins, in a zigzag-like arrangement. Martensite is soft and malleable, and can be easily deformed by de-twinning the structure via an applied strain. At higher temperatures, above a critical temperature of the alloy, Nitinol is austenitic. Austenite is a strong and hard phase of the alloy, exhibiting properties similar to those of titanium, and is characterized by a much more regular crystalline lattice structure. Nitinol alloys can also undergo a phase change as a result of the application of a strain. For example, an element in the austenitic phase can be bent so that at high strain locations the alloy becomes martensitic. If the alloy is designed to have an unstable martensite phase at the operating temperature, removal of the strain results in a reverse transformation that straightens the bending.

FIG. 2 is a graph illustrating a typical phase change temperature hysteresis curve for Nitinol alloys. The percent austenite phase in the alloy is plotted as a function of the temperature, with several important transition temperatures marked. AS and AF indicate the austenite start and finish temperatures, at which the transformation from martensite to austenite starts and is completed, respectively, as the temperature increases. MS and MF indicate the martensite start and finish temperatures, at which the transformation from austenite to martensite begins and is completed, respectively, as the temperature decreases. It is apparent that the two transformations do not occur at the same temperature. Rather, a hysteresis loop exists corresponding to the phase transformation. In addition, an MD temperature exists, indicating the highest temperature at which strain induced martensite can exist, i.e., the temperature above which martensite can not be induced by strain. As would be understood by those skilled in the art, the specific temperatures for a Nitinol alloy are very sensitive to small variations in the alloy's content of nickel, titanium and any other trace elements added to the alloy. Nitinol's properties thus can be tailored for specific applications by controlling the alloy's composition.

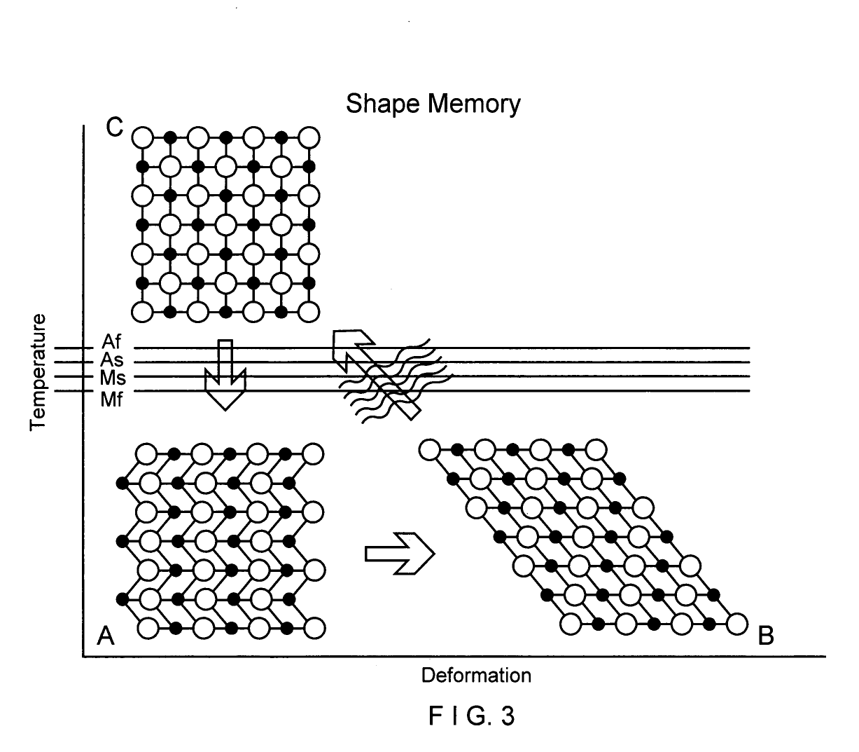

The shape memory and superelasticity properties of Nitinol may be understood in terms of the phase transformations the alloy undergoes under various conditions. As described above, shape memory refers to the ability to restore an originally memorized shape of a plastically deformed Nitinol sample by heating it. FIG. 3 shows a graph of temperature versus deformation illustrating the shape memory effect. An austenitic alloy element has a shape represented by condition C at a temperature above the critical temperature. An element of the alloy is heated to a temperature above the critical temperature and formed into the desired shape. This causes the alloy to memorize the desired shape. As the temperature is lowered below the critical transition temperature, the alloy moves to condition A, in the malleable martensite phase. If a strain is then applied to the alloy element to plastically deform it (e.g., to condition B), as the alloy is martensitic it retains its plastically deformed shape even after the deformation inducing strain has been removed. Then, if the alloy is again heated to a temperature above the critical temperature, a thermoelastic martensitic transformation takes place between state B and state C and the element returns to its memorized shape while regaining the strength and rigidity of the austenitic phase.

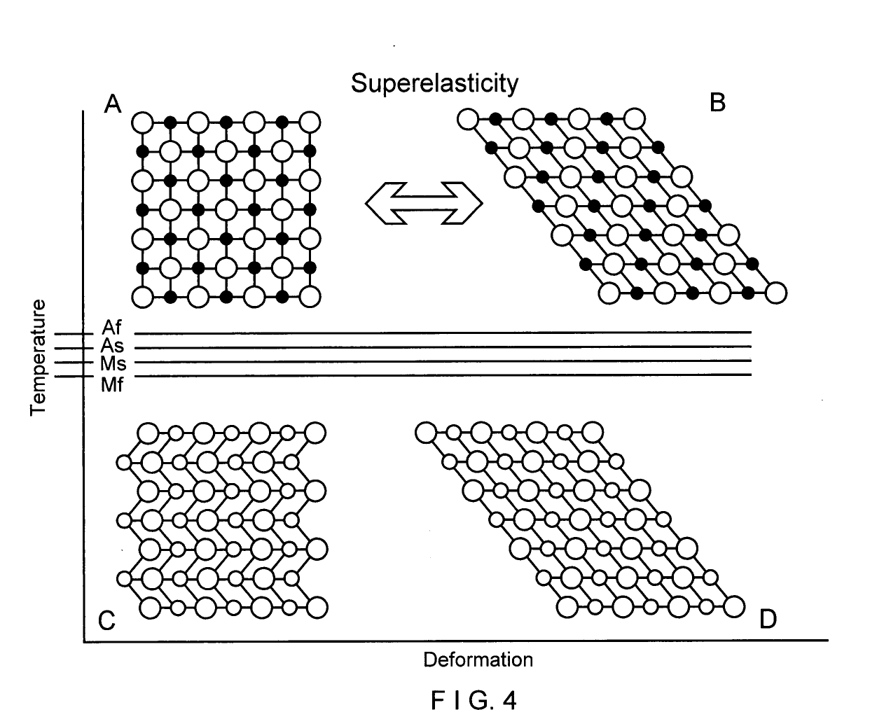

FIG. 4 is a diagram illustrating temperature versus deformation showing the superelasticity property of Nitinol. As discussed above with reference to FIG. 2, an alloy element has a shape that is memorized in state A above the critical temperature in which state the alloy is austenitic. This critical temperature is between the MS and the MD temperatures. When a strain is applied to the alloy element, the element is deformed to the state B in which the alloy element contains large areas of strain-induced martensite. These areas occur primarily at locations at which the highest levels of strain are induced and result in severe deformation that may be unrecoverable in an element of non-shape memory normal material. However, at temperatures at which martensite is not the stable phase of the alloy, as soon as the strain is removed, the alloy reverts to an austenitic state (the A state) and returns to the memorized shape. Superelasticity thus refers to this ability of these alloys while in the austenitic state, to revert to an original shape after severe deformation under strain.

As described above, the shape memory and superelasticity properties of Nitinol and other similar alloys particularly suit them for use in certain medical implants. The shape memory is useful as it allows the implant to convert from a first shape (e.g., a collapsed insertion configuration) to a memorized deployed configuration after being warmed above a critical temperature (e.g., by body heat) while superelasticity is useful to allow the implant to greatly deform while under severe stress in the body, and still return to its original shape.

To obtain the mechanical benefits discussed above, the Nitinol alloy is generally designed to be in the austenitic phase at its operating temperature (i.e., at body temperature), and to be in the martensitic phase at some lower, relatively easy to maintain temperature.

However, it is desired to improve the fatigue life of Nitinol alloys under conditions where strains (particularly repeated strains) imparted thereto are sufficient to cause reversible transformation from austenite to martensite. In addition, it is desired to reduce the formation of fatigue cracks which tend to initiate at the material's surface under bending conditions which may occur in the elements of medical devices. This would be particularly beneficial in implants such as vascular stents and filters which are subject to cyclic strains imparted by blood flow in the vessels in which they are implanted. In addition, many activities such as walking, running etc. can impart repetitive strains to any type of implant.

According to embodiments of the present invention, Nitinol devices are provided that exhibit an increased resistance to fatigue, while retaining their shape memory and superelastic properties. The Nitinol alloy devices according to the invention have an increased ability to withstand cyclic strains, such as may be experienced, for example, within the vascular system.

In the exemplary embodiments, the phase transformation between austenite and martensite in high strain regions of a Nitinol device is prevented. For example, high strain regions may be stabilized in the martensite phase, to eliminate austenite that may incur a damaging reversible phase transformation to martensite in the high strain areas. This result may be obtained by performing certain changes in the chemistry of the high strain regions or by changing internal strains present therein. The remainder of the Nitinol regions, which, depending on the design of a particular implant, may comprise the bulk of the alloy, are left untreated so that they are left in the austenitic phase. Thus, these regions will behave in a manner unchanged with respect to prior Nitinol implants. Thus, large portions of the resulting Nitinol element may retain the original thermal shape memory and superelastic properties that determine the bulk performance of the alloy. These high strain regions may typically comprise surface portions of the alloy elements, since high bending strains tend to take place there. Of course, high strain areas may also be localized along a length of wire or limited in other geographic or functional areas of a device. In these cases, treatments of high stress portions may be applied either to only surface areas in limited portions of the device or to full thickness portions of limited areas of the device. Examples of devices which may benefit from local treatment to minimize the impact of high strains include stents and vena cava filters where most of the deformation occurs in local regions.

The elements formed according to embodiments of the invention exhibit substantial shape memory and superelastic properties and are well suited to the construction of medical devices. At the same time these elements exhibit improved fatigue durability resulting from the surface processing according to the invention. Thus, these elements are especially suited for use in devices requiring extended service life in an environment of cyclic loads. As described above, fatigue failure of Nitinol alloys under bending typically begins when fissures appear at the surface of the metal. As would be understood by those skilled in the art, when an element is bent, the points of greatest strain are surface portions where the radius of curvature is a maximum. In untreated Nitinol, these high strain regions often incur strain induced transformation from austenite to martensite, which may lead to fatigue related failure. Treating a Nitinol alloy in accord with the present invention, extends the service life of cyclically loaded devices by attenuating this failure mechanism.

Since the high strain portions are treated to remain in the martensitic phase even when the device is at a temperature above the critical temperature, applying additional strain to these portions does not result in a phase change. Rather, this additional strain simply results in a deformation of the alloy which remains in the martensitic phase. The damaging reversible strain induced transition from austenite to martensite does not take place, and the life of the Nitinol element is increased. In addition, martensite is more soft and malleable than austenite. Thus an outer layer of martensitic alloy on the outer surface of a Nitinol element reduces the incidence of surface fissures. As would be understood by those of skill in the art, a wide range of implantable medical devices including, for example, vascular and non vascular stents as well as vena cava filters, thrombectomy wires, AAA stent grafts and rotating shafts for IVUS or Rotoblator applications may benefit from treatment according to the present invention. Those of skill in the art will also understand that the initial structural element may include structures other than wire, for example, tubing and sheets of material.

Martensite stabilization of the Nitinol surface may be achieved using any of several processes. For example, the portions may be treated by modifying the chemical composition or applying plastic deformation to produce a surface that remains in the martensite phase even when the alloy is heated above the bulk AS and AF temperatures. Where the portions to be treated include the surface of the device, suitable methods of modifying the surface chemistry include doping the surface (or portions thereof) with pre-selected metals, doping the surface with nitrogen ions, modifying the proportion of titanium and nickel in the surface and forming a cladding on the alloy element with a coating of Nitinol exhibiting the properties desired for the surface. Suitable methods of plastically deforming the surface layer may include, for example, drawing, swaging and extrusion of the alloy, shot peening and other twisting or elongation processes that cause the surface to become martensite stabilized.

More specifically, one way of modifying the surface chemistry of an element formed of a Nitinol alloy involves doping a surface layer thereof with martensite stabilizing elements such as palladium (Pd), platinum (Pt), gold (Au), copper (Cu), hafnium (Hf), zirconium (Zr), tantalum (Ta) and titanium (Ti). The doping may be carried out, for example, by ion implantation, plating, diffusion heat treatment or by composite fabrication. These methods result in a surface portion having a locally increased transformation temperature (AS, AF). Those skilled in the art will understand that these processes may be modified so that the temperatures AS and AF for these treated portions result in an alloy stabilized in the martensite phase under the environmental conditions to which the device is to be subjected.

In ion implantation according to the present invention, the surface is bombarded by high energy ions of a martensite stabilizing element, for example, Pd, Pt, Au, Cu, Hf, Zr, Nb, Ta or Ti. The presence of these elements in the alloy surface modifies the size of the hysteresis loop thereof, and chemically stabilizes the martensite. The elements listed above increase the austenitic phase transformation temperature of the Nitinol alloy, i.e. the AS and AF temperatures, such that at the operational temperature of the element (for example at body temperature) the element's surface is locally stabilized in the martensitic phase, while the bulk or core of the element is austenitic.

Ion implantation may also be used to bombard the alloy element with nitrogen ions. In this process, the ion bombardment results in compressive surface strains, which alone are sufficient to improve the metal's fatigue resistance. In addition, nitrogen ion bombardment eliminates the reversible austenite to martensite phase transformation. Some Ti/N precipitates also may form on the surface as a result of the nitrogen ion bombardment, which tend to increase the fatigue resistance of the metal surface. Nitrogen ion implantation also confers additional beneficial properties to the elements so treated. For example, an improvement in Nitinol corrosion and wear resistance in the in-vivo environment has been noted.

An alternative approach to chemically altering the surface of Nitinol elements involves a surface alloying process. In this case, a pure metal or another alloy is doped into the Nitinol surface. The dopants may include, for example, Pd, Pt, Au, Cu, Hf, Zr or a small amount of Ti. These elements increase the austenitic phase transformation temperature (AS, AF) of the alloy to prevent a phase transformation from austenite to martensite at body temperature (or a different operating temperature) and/or under high strain conditions. For medical devices operating at body temperature, the surface alloying is preferably designed to modify the AS, AF temperatures at the surface to be higher than the body temperature, 37° C. In operation, when implanted in the body, the modified alloy thus remains austenitic at the core, but has a martensitic surface which does not incur phase transformations when subject to strain.

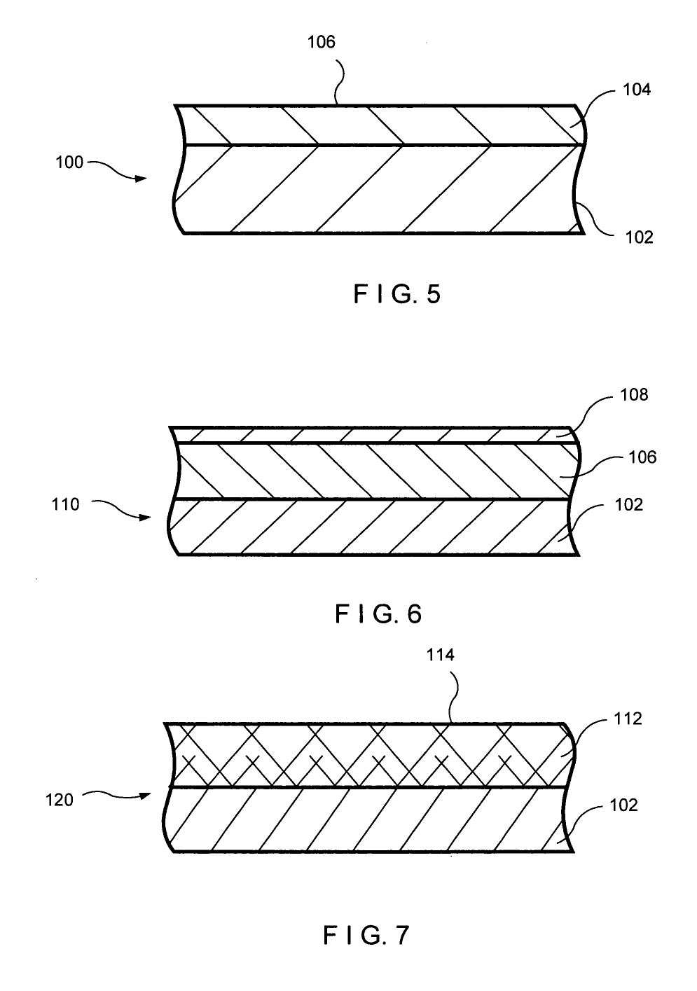

Surface alloying may be accomplished by depositing a desired element on the Nitinol surface by means of different processes, such as PVC, CVD, ion implantation, ion beam assistant deposition, electroplating or composite metal fabrication followed by diffusion heat treatment. FIG. 5 shows an exemplary embodiment of a Nitinol section according to the present invention. Section 100 may be a portion of a Nitinol wire or other element used to construct a medical implant such as a stent or a clot filter. Section 100 has a surface 106 that forms an outer surface of the wire or element, and which is subject to an increased strain when the medical implant is bent. Section 100 includes a core portion 102 formed of primarily untreated alloy, which remains in the austenite phase at the operating temperature of the device. Those skilled in the art will understand that the thickness of the surface layer and the level of doping may be selected to obtain specified properties of section 100.

FIG. 6 shows a different embodiment of a section 110 according to the present invention. In this example, a core portion 102 of the section 110 remains untreated, and comprises a Nitinol alloy in the austenite phase at its operating temperature. A doped outer portion 106 is formed around the core portion 102, e.g., by doping, as described above. The outer portion 106 contains both Nitinol and atoms of the doping material, in a ratio selected to obtain the desired properties of the outer portion 106. A further surface portion 108 may be formed on top of the outer portion 106 to further refine the material properties of the section 110. In this embodiment, the surface portion 108 is formed of substantially only dopant atoms. In the example of surface alloying with a high density metal, such as platinum, a relatively thick layer may be left at the surface to improve visibility under x-ray imaging. In a second example of surface alloying with titanium, a thin but continuous layer of titanium would improve the corrosion resistance of Nitinol. In a further example of surface alloying, a continuous layer of iridium improves the biocompatibility of the device. Those skilled in the art will understand that the chemical composition and size of the surface portion 108 relative to the outer portion 106 may be selected to obtain desired metallurgical properties of the section 110.

In a different exemplary embodiment, the various portions described in the embodiments above may not be distinctly separate, but rather may exhibit a gradual change from a core portion comprising unaltered Nitinol to a surface portion comprising only dopant, or Nitinol with a high percentage of dopant. For example, FIG. 7 shows a section 120 exhibiting this gradually changing metallurgical composition. In this example, a core portion 102 comprises Nitinol, having an unaltered transition temperature. The outer portion 112, on the other hand, comprises a doped alloy of Nitinol with a concentration of the doping material progressively increasing towards the surface 114. Those skilled in the art will understand that the rate of increase of the dopant and the size of the outer portion 112 may be selected to obtain desired properties of the resulting structure. Those skilled in the art will understand that the specific variation in dopant content between the undoped core portion 102 and the doped surface 114 may take innumerable forms, with both gradual changes and stepped changes being possible, depending on the specific properties required of the resulting structure.

In addition, techniques other than vapor deposition and surface alloying may be used to modify the transition temperature of surface portions of a Nitinol alloy element. For example, the nickel to titanium concentration ratio at the surface may be altered. A higher relative concentration of titanium at the surface results in a higher transition temperature, and thus stabilizes the martensite at the surface when exposed to body temperature. A surface Physical Vapor Deposition (“PVD”), Chemical Vapor Deposition (“CVD”), or diffusion coating process of titanium may be used to form this type of surface treated material. A diffusion process in an activated gas may be used to remove nickel from the surface portion fo the alloy, resulting in a higher relative concentration of titanium.

Another approach that may be taken to obtain a Nitinol alloy having different core and surface properties is to utilize a composite fabrication technique. In this process, a layer of Nitinol having a higher AF transition temperature may be clad to Nitinol having a lower AF transition temperature. This construction method results in a composite material having the desired core austenitic properties and surface martensitic properties at the operating temperature. In a different embodiment, a pure metal such as Pd, Pt, Au, Cu, Hf, Zr, Ti or a combination thereof may be clad to the core Nitinol, to obtain a composite alloy having the desired properties. A diffusion heat treatment may be employed to allow the elements in the clad layer to diffuse to the Nitinol surface, and increase the AF transition temperature of that surface. It is also possible that the diffusion treated structure could be cold drawn to obtain the desired final properties or material performance.

In an alternate embodiment of the invention, the surface properties of a Nitinol element may be altered by performing plastic deformation on the surface to introduce compressive strains into the surface material. Those skilled in the art will understand that plastic deformation comprises various mechanical processes conducted without heating the metal such as, for example, shot peening, drawing, swaging and extrusion. Low temperature annealing may be acceptable after plastic deformation. The compressive strains introduced by the plastic deformation induce a phase change of the Nitinol to the martensite phase, resulting in similar dual core/surface properties to that described above in regard to changing the chemical composition of the alloy surface.

As is known in the art, shot peening involves bombarding the surface of the alloy element with a large number of small spheres or pellets. The impact on the surface alters the structure of the alloy near the surface. More specifically, small spherical or other non-cutting particles are used to bombard the sample to plastically deform small portions of the sample surface, and to set up compressive strains in the peened surface. As would be understood by those skilled in the art, the impact of shot peening may be controlled by altering such factors as the size and material of the spheres or pellets used, the nozzle pressure firing the spheres or pellets, the nozzle distance from the sample, and the duration of the process. In one exemplary embodiment, Nitinol wire samples were peened with 30 μm glass beads shot from a three nozzle array located at positions ranging from ⅜″ to 3/16″ from the sample, at pressures of 40 psi and 80 psi. The peening process lasted about 5 sec. Prior to peening, the Nitinol sample in some cases was annealed at 500 C for about 15 minutes. Annealing at 500 C for this period of time is used to “set the shape” of an austenitic material.

Although the shot peening achieved the desired surface modification relative to martensite stabilization, an extremely rough surface was created under these conditions and resulted in reduced beneficial effects thereof. Better results may be obtainable if shot peening is carried out without damaging the surface. For example, traditional peening with smaller particles at lower pressures or laser shock peening would be expected to produce a smoother surface and better fatigue performance

Drawing is another method that may be used to perform plastic deformation on a Nitinol wire. The Nitinol wire may be as-drawn, meaning that it was not annealed after drawing, annealed at a low temperature after drawing or locally deformed after annealing. For example the specific details of any annealing carried out may be modified to accommodate the steps necessary to set the memory shape of the Nitinol alloy. The annealing, for example, may be done at a temperature low enough to maintain the plastic deformation properties, but high enough to set the shape memory of the device. For example, subsequent to drawing a Nitinol wire device, it may be annealed at a low temperature, i.e., 325C, for 7 to 10 minutes, to set the final device shape. The anneal time and temperature may be varied depending on the size of the Nitinol element. In another embodiment, an annealing process, such as 450 C to 550 C for about 13 minutes, may be used to define the austenite shape and local deformation imparted to stabilize martensite in the desired locations.

Testing of the cold drawn and the low temperature annealed Nitinol elements may be carried out to determine the tensile strength, the austenite phase start and finish temperatures (AS, AF) and the fatigue resistance relative to an untreated sample. For example, the transformation temperatures may be determined using a differential scanning calorimeter (DSC), and the fatigue resistance using a rotary bend fatigue test. In one exemplary embodiment, the sample surfaces treated with low temperature annealing and as-drawn exhibited no adverse effect on tensile strength relative to an untreated sample, and did not undergo strain induced martensite transformations. The exemplary samples also exhibited improved fatigue resistance over the untreated elements.

In an alternative embodiment, the starting material may be subjected to heavy plastic deformation or cold worked to stabilize the martensite. The material is formed into the desired shape and areas of lower stress are annealed to set the final device shape leaving high strain areas martensitic through heavy plastic deformation.

The present invention has been described with reference to specific embodiments associated with Nitinol vascular stents and filters. However, other embodiments may be devised that are applicable to other medical devices, without departing from the scope of the invention. Accordingly, various modifications and changes may be made to the embodiments without departing from the broadest spirit and scope of the present invention as set forth in the claims that follow. The specification and drawings are accordingly to be regarded in an illustrative rather than restrictive sense.

1. A flexible device comprising a metallic element including high strain portions and lesser strain portions, wherein the high strain portions are to be subjected to levels of strain during use increased with respect to strain levels in the lesser strain portions, the high strain portions comprising a material which, under predetermined operating conditions to which the device will be subjected when deployed within the body, is stabilized in a martensite phase and the lesser strain portions comprise a material which, under the predetermined operating conditions, is in an austenite phase.

2. The device according to claim 1, further comprising a transition portion disposed between and providing a transition between the high strain and lesser strain portions.

3. The device according to claim 1, wherein the element is formed substantially of Nitinol.

4. The device according to claim 1, wherein an austenite transition temperature of the high strain portion is greater than an austenite transition temperature of the lesser portion.

5. The device according to claim 1, wherein the device is a medical device to be inserted within a living body.

6. The device according to claim 5, wherein an austenite transition temperature of the high strain portion is greater than a body temperature of the living body into which the device is to be inserted.

7. The device according to claim 6, wherein the austenite transition temperature of the high strain portion is greater than 37 C°.

8. The device according to claim 1, wherein the high strain portion is a plastically deformed surface portion of the element.

9. The device according to claim 1, wherein the high strain portion is a surface portion of the element which has been treated with an ion implantation process.

10. The device according to claim 9, wherein the high strain portion includes a portion of a surface of the element into which nitrogen ions have been implanted.

11. The device according to claim 9, wherein the high strain portion includes at least a portion of a surface of the element into which ions of one of Pd, Pt, Au, Cu, Hf, Zr, Nb, Ta and Ti have been implanted.

12. The device according to claim 1, wherein the high strain portion is a doped surface portion of the element.

13. The device according to claim 12, wherein the high strain portion includes at least a portion of a surface of the element in which one of Pd, Pt, Au, Cu, Hf, Zr, Nb, Ta and Ti has been added.

14. The device according to claim 1, wherein a titanium concentration in the high strain portion is greater than a titanium concentration in the lesser strain portion.

15. The device according to claim 8, wherein the plastically deformed portion is one of a shot peened surface and an extruded surface of the element.

16. A medical implant comprising a structural element defining a shape of at least a portion of the implant, a super-elastic core portion of the element being primarily formed of Nitinol which, at body temperature, is in a substantially austenitic phase and a fatigue resistant surface portion primarily formed of Nitinol which, at body temperature, is substantially Martensite phase stabilized.

17. The implant according to claim 16, wherein the element is one of a wire, tubing and a sheet.

18. The implant according to claim 16, wherein at least a portion of a surface of the element is plastically deformed.

19. The implant according to claim 18, wherein the plastically deformed portion of the surface is one of shot peened and low temperature extruded.

20. The implant according to claim 16, wherein the fatigue resistant surface portion is treated with an ion implantation process.

21. The implant according to claim 16, wherein the fatigue resistant surface portion is doped with one of Pd, Pt, Au, Cu, Hf, Zr, Nb, Ta and Ti.

22. The implant according to claim 16, wherein the fatigue resistant surface portion is a cladding layer formed around the super-elastic core portion.

23. The implant according to claim 19, wherein the fatigue resistant surface portion has a ratio of nickel and titanium modified with respect to that of the super-elastic core portion.

24. A method of forming an element of a medical device comprising the steps of:
forming an element of the device of Nitinol;
impressing a memorized shape on the element, wherein the memorized shape is a shape the element is to assume when in an operational configuration; and
treating a high strain portion of the element so that the high strain portion is substantially Martensite phase stabilized under expected operating conditions of the device, wherein untreated portions of the element are in a substantially austenitic phase under the expected operating conditions.

25. The method of claim 24, wherein the high strain portion is substantially Martensite phase stabilized by plastic deformation.
